(19) United States
(12) Patent Application Publication
(10) Pub. No.: US 2005/0149196 A1
(43) Pub. Date: Jul. 7, 2005

(54) Artificial spinal disk replacement device with rotation limiter and lateral approach implantation method

(75) Inventors: James F. Zucherman, San Francisco, CA (US); Ken Y. Hsu, San Francisco, CA (US)

Correspondence Address: FLIESLER MEYER, LLP, FOUR EMBARCADERO CENTER, SUITE 400, SAN FRANCISCO, CA, 94111, US

(73) Assignee: St. Francis Medical Technologies, Inc., Alameda, CA (US)

(21) Appl. No.: 11/004,463

(22) Filed: Dec. 3, 2004

Related U.S. Application Data
(60) Provisional application No. 60/534,734, filed on Jan. 7, 2004.

(51) Int. Cl. A61F 2/44

(52) U.S. Cl. 623/17.14; 623/17.15

(57) ABSTRACT

An intervertebral spinal disk replacement device which can be implanted via a lateral approach includes a first end plate, a second end plate, an articulating element and a limiter. The articulating element and limiter, which are situated between the two end plates, are integral to the second end plate. The articulating element with an articulating surface that is slidably positioned on the support surface of the first end plate and the limiter that protrudes from the upper surface of the second end plate, with the limiter being confined with a cavity within the first end plate. The degree of rotational movement of the first end plate relative to the second end plate is determined by the range of motion the limiter exhibits within the cavity. The implant may include keels that extend in a lateral direction with respect to the vertebral bodies on which the end plates abut.

### PRIORITY CLAIM

This application claims priority to U.S. Provisional Patent Application No. 60/534,734 entitled: ARTIFICIAL SPINAL DISK REPLACEMENT DEVICE WITH ROTATION LIMITER AND LATERAL APPROACH IMPLANTATION METHOD, by Zucherman et al., filed Jan. 7, 2004 (Attorney Docket No. KLYCD-05001US0).

### FIELD OF THE INVENTION

This invention relates to an artificial vertebral disk replacement and method.

### BACKGROUND OF THE INVENTION

The spinal column is a biomechanical structure composed primarily of ligaments, muscles, vertebrae and intervertebral disks. The biomechanical functions of the spine include: (1) support of the body, which involves the transfer of the weight and the bending movements of the head, trunk and arms to the pelvis and legs, (2) complex physiological motion between these parts, and (3) protection of the spinal cord and nerve roots.

As the present society ages, it is anticipated that there will be an increase in adverse spinal conditions which are characteristic of older people. Pain associated with such conditions can be relieved by medication and/or surgery. Of course, it is desirable to eliminate the need for major surgery for all individuals and in particular for the elderly.

More particularly, over the years, a variety of intervertebral implants have been developed in an effort to relieve the pain associated with degenerative and dysfunctional disk conditions. For example, U.S. Pat. No. 4,349,921 to Kuntz discloses an intervertebral disk prosthesis.

U.S. Pat. No. 4,714,469 to Kenna discloses a spinal implant that fuses vertebrae to the implant. The implant has a rigid body that fits between the vertebra with a protuberance extending from a vertebral contacting surface and extends into the vertebral body.

U.S. Pat. No. 5,258,031 to Salib et al. discloses another prosthetic disk with a ball that fits into a socket.

U.S. Pat. Nos. 5,425,773 and 5,562,738 are related patents to Boyd et al. that disclose a disk arthroplasty device for replacement of the spinal disk. A ball-and-socket are provided to enable rotation.

U.S. Pat. No. 5,534,029 to Shima discloses an articulated vertebral body spacer with a pair of upper and lower joint pieces inserted between the vertebra. An intermediate layer is provided to allow for movement between the upper joint piece and the lower joint piece.

U.S. Pat. No. 5,782,832 to Larsen et al. discloses a two-piece ball-and-socket spinal implant with upper and lower plates for insertion within the intervertebral space.

U.S. Pat. No. 6,156,067 to Bryan et al. discloses a prosthesis having two plates with a nucleus there between.

Even given the above devices there needs to be developed enhanced implants for alleviating such conditions, and for restoring natural movement of the spine.

### BRIEF DESCRIPTION OF THE DRAWINGS

FIGS. 1A and 1B are the front elevational and side elevational views, respectively, of an embodiment of the assembled implant of the invention.

FIGS. 1C, 1D, and 1E are the plan view of the first or outer surface of the first end plate, the plan view of the second or inner surface of the first end plate, and a perspective view of the first end plate, respectively, of the implant.

FIGS. 1F and 1G are the plan view of the first or outer surface of the second end plate, and a perspective view of the second end plate, respectively, of the implant.

FIG. 1H shows the cross-sectional view of the implant along the 1H-1H line of FIG. 1A.

FIG. 1I shows the cross-sectional view of the implant along the 1I-1I line of FIG. 1H.

FIG. 1J is back elevational view of the assembled implant.

FIG. 2 is a block diagram showing the method steps for the lateral implantation of an embodiment of the disclosed the disclosed implant.

### DETAILED DESCRIPTION OF THE PREFERRED EMBODIMENTS OF THE INVENTION

Embodiments of the present invention are directed to an intervertebral implant for alleviating discomfort associated with the spinal column. The implant is characterized by having a first end plate and a second end plate, wherein the latter has a curved or convex articulating element and a limiter. The articulating element, which also functions as a weight bearing member, rests within a recess or groove that serves as a support surface of the first end plate and the limiter is positioned within a ridge or cavity that is also formed on the surface of the first end plate. The articulating element enables the plates to move relative to each other, but the degree of rotational movement is confined within a desired range by the cooperation of the limiter and the walls of the cavity.

The following description is presented to enable any person skilled in the art to make and use the invention. Various modifications to the embodiments described will be readily apparent to those skilled in the art, and the principles defined herein can be applied to other embodiments and applications without departing from the spirit and scope of the present invention as defined by the appended claims. Thus, the present invention is not intended to be limited to the embodiments shown, but is to be accorded the widest scope consistent with the principles and features disclosed herein. To the extent necessary to achieve a complete understanding of the invention disclosed, the specification and drawings of all patents and patent applications cited in this application are incorporated herein by reference.

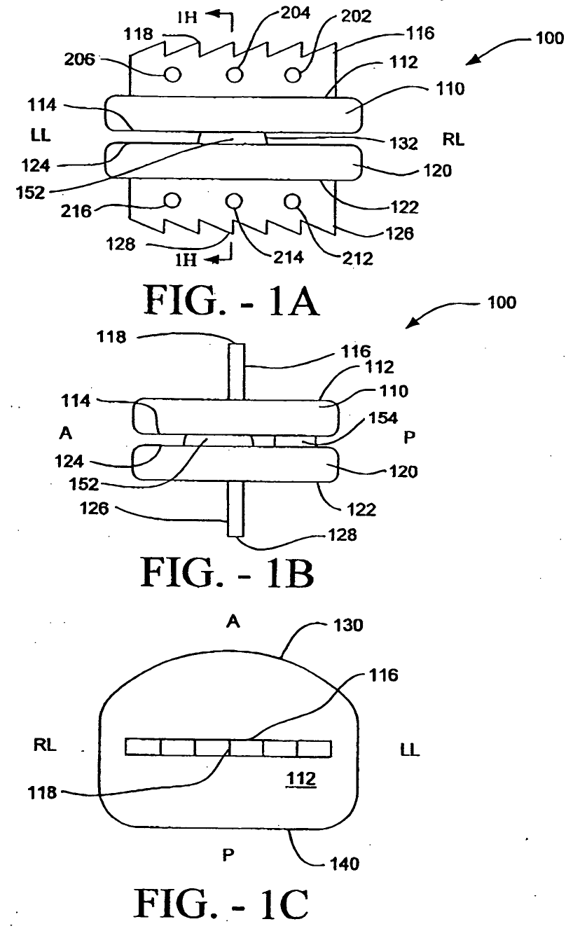

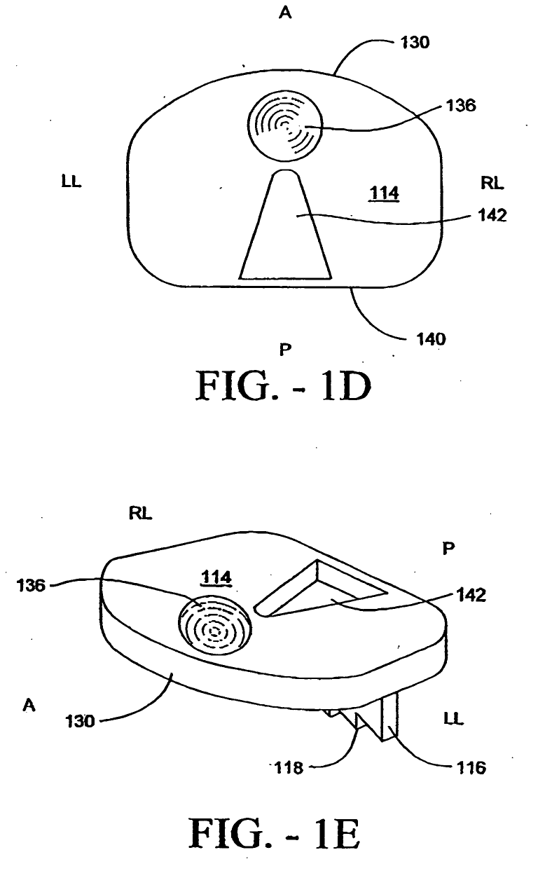

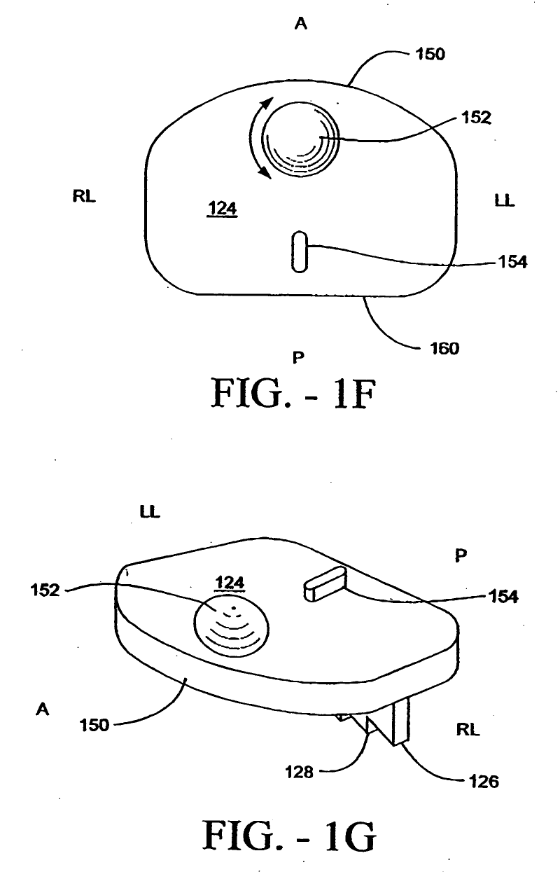

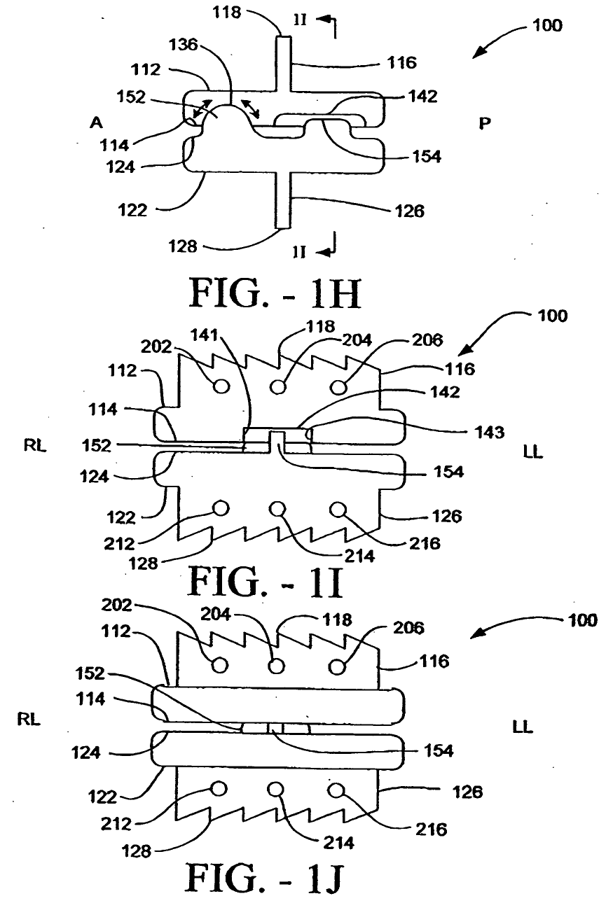

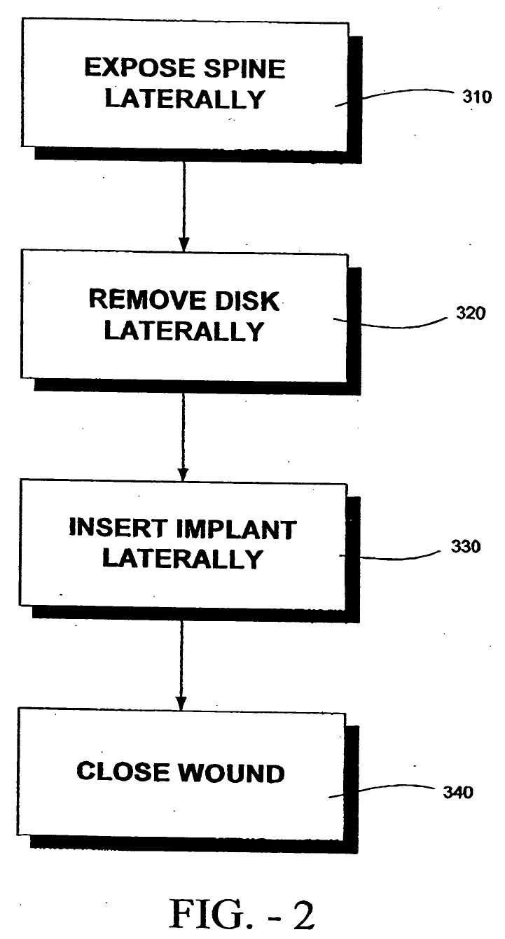

FIGS. 1A, 1B and 1C show an embodiment of the assembled implant 100 which includes a first end plate 110 that is configured to mate with a first vertebra and a second end plate 120 that is configured to mate with a second vertebra. The designations, “A” for anterior, “P” for posterior, “RL” for right lateral, and “LL” for left lateral are given in the drawings for spatial orientation. These designations give the relationship of all faces of the implant from the superior perspective, i.e., looking down the axis of the spine. The first or upper end plate 110 has a first or outer surface 112 from which a keel 116 extends. The first surface 112 abuts the vertebra body when the implant implanted. The keel 116 extends into the vertebra body to anchor the implant into position. The keel 116 includes teeth 118 that assists in keeping the keel 116 in place once the implant is positioned between the vertebra bodies. As will be further described herein, the second or inner surface 114 of the first end plate defines (i) a recess which accommodates an articulating surface and (ii) a limiter cavity or channel that accommodates a limiter. The second surface 114 can form a planar surface that is parallel to the first surface 112, or can form a planar surface that is unparallel to the first surface 112.

In one embodiment, when implant 100 is inserted between vertebrae, the first keel 116 extends longitudinally across the first surface 112, about perpendicular to the sagittal plane of the spine. In another embodiment, the first keel 116 extends longitudinally only partially across the first surface 112, about perpendicular to the sagittal plane of the spine. The teeth in the two embodiments with complete or partial extension of the keel across the first surface 112 of the first end plate 110 point toward the right lateral face of implant 100 when the embodiment is meant to be put into a slot in a vertebral body from the right lateral approach to the spine. This orientation is shown in FIG. 1A, for example. Alternatively, the teeth 118 point toward the left lateral face of implant 100 when the embodiments are meant to be put into a slot in a vertebral body from the left lateral approach to the spine.

The upper surface 112 is positioned so that the first keel 116 extends into the vertebral body to anchor implant 100 into position and is perpendicular to the median sagittal plane of the spine, in which extension and flexion occur. The first keel 116 in this orientation offers substantial stability during extension and flexion for implant 100 inserted between the vertebrae of a patient. Additionally, the first keel 116 is aligned with and supports the lateral axis of articulation of implant 100 perpendicular to the sagittal plane of the spine.

The second or lower end plate 120 has a first or outer surface 122 from which a second keel 126 extends with a second set of teeth 128. In one embodiment, when implant 100 is inserted between vertebrae, the second keel 126 is about perpendicular to the sagittal plane of the spine in which extension and flexion occur. The second keel 126 in this orientation offers substantial stability during extension and flexion for implant 100 inserted between the vertebrae of a patient. Additionally, the second keel 126 in this embodiment is aligned with and supports the lateral axis of articulation of implant 100 perpendicular to the sagittal plane of the spine.

As described above for the first or upper end plate 110, in one embodiment, the second keel 126 extends longitudinally across the outer surface 122, while in another embodiment, the second keel 126 extends longitudinally partially across the outer surface 122. Similarly, the teeth in the two embodiments with complete or partial extension of the keel across the outer surface 122 of the lower end plate 120 point towards the left lateral face of implant 100 when the embodiment is meant to be put into a slot in a vertebral body from the left lateral approach to the spine. Alternatively, the teeth 128 point towards the right lateral face of implant 100 when the embodiments are meant to be put into a slot in a vertebral body from the right lateral approach to the spine.

The second or inner surface 124 can form a planar surface that is parallel to the first or outer surface 122, or can form a planar surface that is not parallel to the outer surface 122.

Protruding from the first surface 124 of the second end plate 120 are articulating element 152, which has an articulating surface, and limiter 154. The articulating surface 132, which preferably has a convex exterior contour, such as a ball contour, allows the first end plate 110 and second end plate 120 to pivot and/or rotate relative to each other. The limiter 154 is preferably configured as an elongated ridge which functions to limit the rotational movement of the plates to within a desired range. The articulating element 152 is integral with the inner surface 124 of the second end plate 120 which means that articulating element is permanently attached or connected to the inner surface. The articulating element 152 can preferably be formed integrally with the inner surface 124 or it can be attached thereto, such as by welding. The limiter 154 is also integral with the inner surface 124.

The outer surface 112 of the first end plate 110 can be parallel to the outer surface 122 of the second end plate 120 when implant 100 is assembled and is in a neutral position (i.e., the position where the first end plate 110 has not rotated relative to the second end plate 120). Alternatively, the outer surface 112 of the first end plate 110 can be non-parallel to the planar surface of the outer surface 122 of the second end plate 120 when implant 100 is assembled and in a neutral position. This non-parallel orientation of the first end plate 110 and the second end plate 120 allows the plates to pivot to a greater degree with respect to each other. Additionally, other factors such as the height and position of the articulating surface 132 can also be adjusted in order to increase the degree that the first end plate 110 and the second end plate 120 can pivot relative to each other.

The embodiment shown in FIG. 1A illustrates the first and second keels 116,126, which include ports 202, 204, 206 and 212, 214, 216, respectively, that facilitate bone ingrowth. For example, bone from the vertebral bodies can grow thorough the ports and aid in securing the first and second keels 116,126 and the implant 100 with respect to the vertebral bodies. In addition, surfaces defined by the first and second keels 116,126 and the outer surfaces 112, 122 of implant 100 can be roughened in order to promote bone ingrowth into these defined surfaces of implant 100. In another embodiment the ports, the first and second keels 116,126, and the outer surfaces 112, 122 of implant 100 can be coated with materials that promote bone growth such as for example bone morphogenic protein, BMP, or structural materials such as hyaluronic acid, HA, or other substance which promotes growth of bone relative to and into the keel, keel ports, and other external surfaces of the implant 100.

When implant 100 is inserted between vertebrae the planar surfaces corresponding to the outer surfaces 112, 122 and the inner surfaces 114, 124 of the first and second end plates 110, 120 lie within, or substantially within, the axial plane of the body of the patient. Similarly, the first and second keels 116,126 are aligned in the axial plane, or perpendicular to the sagittal plane of the vertebrae.

As shown in FIG. 1B, in a preferred embodiment, the implant includes two keels 116 and 126 that are connected to the first end plate 110 and second end plate 120, respectively; the keels penetrate into the vertebral bodies. It is understood that the implant may include just one of the two keels or alternatively, either end plate can be connected to more than one keel. In addition, while the keels are shown to be substantially perpendicular to outer surfaces 112 and 122, respectively, it is also understood that any of the keels that is connected to the first end plate 110 or second end plate 120 may not be perpendicular, that is, the keel extends at an angle relative to outer surface 112 or 122. Furthermore, as shown in FIGS. 1B and 1C, keels 118 and 126 are substantially aligned with the articulating element 152 in that the keels are positioned above and below the articulating element to provide support thereof.

As shown in FIG. 1C, the anterior surface 130 of the first end plate 110 preferably has a convex contour whereas the posterior surface 140 preferably has a substantially planar contour.

As illustrated in FIGS. 1D and 1E, the inner surface 114 of the first end plate 110 defines recess 136 and limiter cavity 142. The recess 136 has an exterior contour which conforms the shape of the articulating element 152 of the second end plate 120. The complementary configurations of the recess and the articulating element allow the implant to simulate the natural motion of the spine. In a preferred embodiment, the articulating element 152 has a raised articulating surface that is configured as a hemisphere and the corresponding recess 136 has a matching exterior contour shaped as a symmetrical circular cavity or concave cavity. As further described herein, the recess 136 covers only a portion of the surface area of the articulating element 152 at any given time. In this fashion, as the recess 136 traverses or rolls over different areas of the articulating surface, the first end plate 110, in turn, moves relative to the second end plate 120.

The limiter cavity 142 is a depression formed on the inner surface 114 of the first or upper end plate 110. The limiter cavity 142, in association with the limiter 154 that projects from the inner surface 124 of the second or lower end plate 120, functions to confine the rotational or twisting motion of the first end plate 110 relative to the second end plate 120 to a prescribed range. The limiter cavity.142 defines the region or zone in which the limiter 154 is allowed to move. This in turn limits the rotational movement of the first and second end plates 110, 120 relative to each other.

As shown in FIGS. 1D and 1E, in this embodiment, the limiter 142 has an isosceles triangular configuration with a base and two equal sides. The range of rotational movement is determined by the angle between the two equal sides. Typically, this angle will range from 10 to 90 degrees and preferably will range from 25 to 75 degrees.

As shown in FIGS. 1F and 1G, the second end plate 120 includes an inner surface 124 on which the articulating element 152 and the limiter 154 are formed. As illustrated, in this embodiment, the articulating element 152 is configured as a half sphere with a circular perimeter on the inner surface 124. The exterior surface contour of the articulating element 152 and recess 136 (FIG. 1F) should be substantially complementary so that the two surfaces can slide smoothly over each other. The second end plate preferably has a convex anterior surface (A) 150 and a substantially planar posterior surface (160) (P).

The limiter 154 interacts with the two sides of the limiter cavity 142 to define the range of the rotational movement of the end plates 110, 120 relative to each other. The size and configuration of the limiter 154 should be sufficient to withstand the stresses generated by the impact to the sides of the limiter cavity 142. In a preferred embodiment, the limiter 154, as illustrated, is an elongated member with a length that is slightly shorter than the length of a side of the limiter cavity 142. When the limiter 154 contacts either side of the limiter cavity 142, it is preferred that a substantial portion of the surface of the limiter 154 comes into contact with the limiter cavity 142. In other words, the surface of the limiter 154 is preferably flushed with the corresponding side on the limiter cavity 142. This allows most of the force created on impact to be dissipated throughout the limiter 154 rather than to have the force concentrated at a small area or a single point on the limiter 154.

As illustrated in FIG. 1H, the height of the articulating element 152, as measured from the inner surface 124, is greater than the depth of the corresponding cavity of the recess 136, as measured from the inner surface 114. In this fashion, the first end plate 110 and the second end plate 120 can pivot or rotate relative to each other. As shown in FIG. 1F, the implant 110 affords twisting or torsional movement of the second end plate 120 about an axis that is about parallel to the axis of the spine. Thus, the implant allows the spine to have movement in three orthogonal degrees of freedom namely (1) forward (flexion) and backward (extension) bending movement, (2) lateral side to side bending, and (3) twisting movement.

In order to simulate the natural movement of the spine, the implant preferably includes the limiter 154 that restrains the range of twisting motion. As illustrated in FIGS. 1I and 1J, the limiter 154 is flanked by sides 141 and 143 of cavity 142. Thus, when first end plate 110 is twisted relative to the second end plate 120, one of the sides of the limiter cavity 142 will come into contact with one of the limiter.

As shown in FIG. 1H, which illustrates the implant in a neutral position, i.e., the position where the first end plate 110 has not rotated relative to the second end plate 120, the upper surface of the limiter 154 does not touch the lower surface-of the limiter cavity 142. This permits the flexion and extension motions. As is apparent, the distance of the gap or clearance between the limiter 154 and the limiter cavity 142 is also another factor that determines the degree of bending. The greater the distance, the higher degree of bending movement possible. Thus, to enable a person to experience a high level of flexion and extension with the implant 100, this gap distance and the height differential between the articulating element 152 and corresponding cavity 142 should be maximized. While the level of movement can be tailored by appropriate design of the components, it is understood the intervertebral implant functions in conjunction with the unaffected (or natural) structures of the spinal column. For example, the annulus fibrosis along with the facet joints restrict the torsional motion or twisting between vertebrae.

In constructing the first end plate 110, the keel 116 can be formed integrally with the first end plate or can be attached using conventional techniques such as molding or machining. Similarly, with respect to the second end plate 120, the keel 128 can be formed integrally or can be attached.

It is to be understood that the embodiments of the disclosed implant can be made of medical grade titanium, stainless steel or cobalt chrome. Other materials that have appropriate structural strength and that are suitable for implantation into a patient can also be used.

Alternatively, the components of the implant can be made out of a polymer, and more specifically, the polymer is a thermoplastic with the other components made of the materials specified above. Still more specifically, the polymer is a polyketone known as polyetheretherketone (PEEK). Still more specifically, the material is PEEK 450G, which is an unfilled PEEK approved for medical implantation available from Victrex of Lancashire, Great Britain. (Victrex is located at www.matweb.com or see Boedeker www.boedeker.com). Other sources of this material include Gharda located in Panoli, India (www.ghardapolymers.com). The components can be formed by extrusion, injection, compression molding and/or machining techniques. This material has appropriate physical and mechanical properties and is suitable for carrying and spreading the physical load between the spinous process. Further in this embodiment, the PEEK has the following additional approximate properties:

| Property | Value | |
|---|---|---|
| Density | 1.3 | g/cc |
| Rockwell M | 99 | |
| Rockwell R | 126 | |
| Tensile Strength | 97 | Mpa |
| Modulus of Elasticity | 3.5 | Gpa |
| Flexural Modulus | 4.1 | Gpa |

It should be noted that the material selected may also be filled. For example, other grades of PEEK are also available and contemplated, such as 30% glass-filled or 30% carbon-filled, provided such materials are cleared for use in implantable devices by the FDA, or other regulatory body. Glass-filled PEEK reduces the expansion rate and increases the flexural modulus of PEEK relative to that which is unfilled. The resulting product is known to be ideal for improved strength, stiffness, or stability. Carbon-filled PEEK is known to enhance the compressive strength and stiffness of PEEK and lower its expansion rate. Carbon-filled PEEK offers wear resistance and load carrying capability.

The implant components can also be comprised of polyetherketoneketone (PEKK). Other material that can be used include polyetherketone (PEK), polyetherketoneether-ketoneketone (PEKEKK), and polyetheretherketoneketone (PEEKK), and, generally, a polyaryletheretherketone. Further, other polyketones can be used as well as other thermoplastics.

Reference to appropriate polymers that can be used in the spacer can be made to the following documents, all of which are incorporated herein by reference. These documents include: PCT Publication WO 02/02158 A1, dated Jan. 10, 2002, entitled “Bio-Compatible Polymeric Materials;” PCT Publication WO 02/00275 A1, dated Jan. 3, 2002, entitled “Bio-Compatible Polymeric Materials;” and, PCT Publication WO 02/00270 A1, dated Jan. 3, 2002, entitled “Bio-Compatible Polymeric Materials.”

In operation, implant 100 enables a forward bending movement and a rearward bending movement by sliding the upper end plate 110 forward and backward over the articulating surface 152 relative to the lower end plate 120.

The implant 100 enables a right lateral bending movement and a left lateral bending movement by sliding the upper end plate 110 side-to-side over the articulating surface 152 relative to upper end plate 110. This movement is shown as rotation about the axis defined by the articulating surface 152 in FIG. 1F.

FIG. 6 is a block diagram showing the basic steps of the method of laterally inserting the implant 100. First the spine is exposed through a lateral access 310, then the intervertebral disk or portions thereof is removed laterally 320 if necessary. This can be accomplished by exposing an annulus that is between first and second adjacent vertebrae and then removing at least a portion of the fibrous tissue from the annulus. The implant is then inserted laterally 330 in the intervertebral space between two vertebrae and the wound is then closed 340. This procedure can be followed for either a left lateral approach or right lateral approach. For a left lateral approach, the teeth 118, 128 of upper and lower keels 116, 126 would be pointed towards the left lateral face of the device in order to aid in retaining implant 100 in place. For a right lateral approach, the teeth would point towards the right lateral face of the device.

Additional steps, such as cutting channels into the vertebral bodies to accept the first and second keels 116,126 of the first and second end plates 110, 120 can also be performed without departing from the scope of what is disclosed.

The foregoing description of embodiments of the present invention has been provided for the purposes of illustration and description. It is not intended to be exhaustive or to limit the invention to the precise forms disclosed. Many modifications and variations will be apparent to the practitioner skilled in the art. The embodiments were chosen and described in order to best explain the principles of the invention and its practical application, thereby enabling others skilled in the art to understand the invention and the various embodiments and with various modifications that are suited to the particular use contemplated. It is intended that the scope of the invention be defined by the following claims and its equivalence.

1. An intervertebral implant adapted to be inserted between adjacent vertebrae comprising:
a first end plate with a first inner surface; and
a second end plate with a second inner surface opposing the first inner surface, wherein the first inner surface defines a support surface and a limiting ridge with a first ridge side and a second ridge side, and wherein the second inner surface defines (i) an articulating surface that is slidably positioned on the support surface of the first end plate and (ii) a limiter that protrudes from the second inner surface, with the limiter being confined between the first ridge side and the second ridge side.

2. The intervertebral implant of claim 1 wherein the degree of rotational movement of the first end plate relative to the second end plate is determined by the range of motion the limiter exhibits between the first ridge side and the second ridge side.

3. The intervertebral implant of claim 2 wherein the degree of flexion movement of the first end plate relative to the second end plate is proportional to a gap distance between the limiter and the limiting ridge when the first and second end plates are in a neutral position where the first and second end plates are not flexed relative to each other.

4. The intervertebral implant of claim 1 wherein the articulating surface has a convex exterior surface that is in contact with the support surface.

5. The intervertebral implant of claim 4 wherein the support surface has a concave exterior surface.

6. An intervertebral implant comprising:
a first end plate and a second end plate;
a first articulation surface extending from one of the first end plate and the second end plate;
a first articulation receiving surface defined on the other of the first end plate and the second end plate;
a first motion limiter extending from one of the first end plate and the second end plate; and
a first motion limiter receiving surface defined on the other of the first end plate and the second end plate.

7. The implant of claim 6 wherein said first articulation surface and the first motion limiter both extend from the first end plate.

8. The implant of claim 6 wherein said first articulation surface and the first motion limiter both extend from either the first end plate or the second end plate.

9. The implant of claim 6 including the first end plate including a first keel adapted to engage a first vertebral body and the second end plate includes a second keel adapted to engage a second vertebral body.

10. The implant of claim 6 wherein said first articulation receiving surface defines a first recess and the first motion limiter receiving surface defines a second recess.

11. The implant of claim 6 wherein said first recess is hemispherical in shape and the second recess is triangular in shape.

12. The implant of claim 6 wherein said first articulation surface is hemispherical in shape and said first motion limiter is planar in shape.

13. The implant of claim 6 wherein said first articulation surface is hemispherical in shape.

14. In an intervertebral implant the improvement including an articulation mechanism and a motion limiting mechanism extending and defined between first and second end plates and wherein said articulation mechanism includes a first extension from one of the first and second plates, which first extension is received in a first recess in the other of the first and second plates, and the motion limiting mechanism includes a second extension from one of the first and second plates, which second extension is received in a second recess in the other of the first and second plates.

15. In an intervertebral implant the improvement including an articulation mechanism and a twisting motion limiting mechanism.
